(19) United States
(12) Patent Application Publication
(10) Pub. No.: US 2025/0025339 A1
(43) Pub. Date: Jan. 23, 2025

(54) METHOD FOR PROVIDING CONTROL DATA FOR AN OPHTHALMOLOGICAL LASER OF A TREATMENT APPARATUS, CONTROL DEVICE THEREFOR, TREATMENT APPARATUS, COMPUTER PROGRAM PRODUCT AND COMPUTER-READABLE MEDIUM

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/772,698

(22) Filed: Jul. 15, 2024

(30) Foreign Application Priority Data
Jul. 19, 2023 (DE) 10 2023 119 081.4

(51) Int. Cl. A61F 9/008

(52) U.S. Cl. CPC A61F 9/008; A61F 2009/00851; A61F 2009/00872; A61F 2009/00878

(57) ABSTRACT

The invention relates to a method for providing control data for an ophthalmological laser (12) of a treatment apparatus (10) for treating a human or animal eye (16), wherein the method comprises the following steps performed by a control device (18): setting an area (14) to be irradiated on or in a cornea of the eye (16) to be treated for performing an irradiation treatment with the laser (12), setting a virtual irradiation mask (50) in a mask plane (40), wherein the area (14) to be irradiated is situated within the virtual irradiation mask (50) in a perpendicular projection onto the mask plane (40), and providing control data for the laser (12) by the control device (18), wherein the control data includes coordinates of the area (14) to be irradiated such that the laser (12) emits laser pulses to the area (14) to be irradiated during the treatment.

### FIELD

The invention relates to a method for providing control data for an ophthalmological laser of a treatment apparatus, to a control device therefor, to a treatment apparatus, to a computer program product and to a computer-readable medium.

### BACKGROUND

Treatment apparatuses and methods for controlling ophthalmological lasers for correcting or treating an optical visual disorder and/or pathologically or unnaturally altered areas of the cornea are known in the art. Therein, a pulsed laser and a laser scanner device or beam deflection device can for example be formed such that laser pulses cause a photodisruption and/or photoablation in a focus situated within an organic tissue to remove or cauterize the tissue from the cornea.

Herein, the phototherapeutic keratectomy (PTK) has been established as a safe and effective method for treating anterior corneal pathologies. The recurrent erosion syndrome, the Salzmann degeneration and the corneal scarring and others belong to these pathologies, which can be diffuse, but are often focal. The current PTK technology in the Salzmann degeneration includes manually peeling the corneal lesion and then smoothing the underlying irregular stroma as needed with small laser spots. The smoothing can be associated with the use of a masking liquid. The current technology does not allow an accurate matching between the laser ablation profile and the lesion, wherein lesions are usually not perfectly round. In addition, laser appliances in the prior art do not allow the selection of very small laser spots. Surgeons usually circumvent these restrictions by the use of masking liquids.

### SUMMARY

It is the object of the invention to improve a laser treatment method and in particular a phototherapeutic keratectomy method and to provide a method which can provide more suitable irradiation parameters for an ophthalmological laser.

This object is solved by the examples provided herein. Advantageous developments are disclosed in the dependent claims, the present description as well as the figures.

The invention is based on the idea to set an irradiation mask with any shape for an irradiation of a human or animal eye, wherein the shape follows pathological alterations or other features of the eye. Areas within the irradiation mask can be irradiated with the laser in the subsequent irradiation, areas outside of the irradiation mask cannot be irradiated.

By the invention, a method for providing control data for an ophthalmological or eye surgical laser of a treatment apparatus for treating a human or animal eye is provided, wherein the method comprises the following steps, which are performed using or by a control device.

In the method, setting an area to be irradiated on or in a cornea of the eye to be treated is effected to perform an irradiation treatment with the laser. The area to be irradiated can be an area, which is used for ablation, for generation of microbubbles, for generation of an incision surface in particular within the cornea, for cauterizing, for cross-linking, for visual disorder treatment, for tissue alteration etc.

Further, setting a virtual irradiation mask in a mask plane is effected. Herein, the mask plane represents a virtual plane, which is perpendicular to an irradiation direction of the laser onto the cornea in a rest position of a beam deflection device of the laser or is perpendicular to an optical axis of the laser in the rest position of the laser.

Herein, the area to be irradiated is within the virtual irradiation mask in a perpendicular projection onto the mask plane, that is in a projection perpendicular onto the mask plane or along the irradiation direction of the laser (z-axis). Herein, the irradiation direction of the laser (z-axis) is an irradiation direction of the laser in a rest position of the beam deflection device. This means, an area, which is outside of the virtual irradiation mask in the perpendicular projection onto the mask plane, is certainly not irradiated with the laser in the subsequent irradiation and thus is not encompassed in the area to be irradiated.

Further, providing control data for the laser using the control device is effected in the method, wherein the control data includes coordinates of the area to be irradiated or coordinates of irradiation positions of the area to be irradiated, such that the laser can emit laser pulses to the area to be irradiated during the treatment. Thus, the control data can be used to deflect the laser using a beam deflection device. The setting of irradiation positions within the area to be irradiated can be effected in a predefined pattern according to known methods such that a density of laser pulses in the area to be irradiated is for example constant or is adapted to a thickness of the area to be ablated. Herein, the area to be irradiated can for example be a curved surface or a 3D volume, the area to be irradiated can for example be a pathologically altered area and the area to be irradiated can be determined depending on examination data of the cornea. The virtual irradiation mask in the mask plane is, for example, a geometric shape like a rectangle or triangle, a circular segment or a circular sector, and the control data can be set by the virtual irradiation mask. Data for a static cyclotorsion correction for a treatment can be added to the control data.

By the invention, the advantage arises that an area to be irradiated can be specifically treated using the irradiation mask. Herein, the area to be irradiated can, for example, be situated decentered and can have any shape. By the use of the virtual irradiation mask, it can be ensured that an overall lesion can be removed, and therein adjacent tissue not affected is impaired as little as possible or an unnecessary removal of tissue is avoided. Thus, a faster recovery of the eye and of the vision also arises and a lower influence on a refraction of the eye, in particular in case of peripheral lesions, also results. Compared to a conventional PTK treatment in the prior art, which incorporates the entire diameter of the optical zone, the invention allows a defined treatment of the pathology and avoids unnecessary epithelial and stromal ablations, whereby better visual results can be achieved. Moreover, a focal PTK is a good alternative to the conventional PTK in cases with recurrent erosions without associated focal lesions if the erosion region can be preoperatively determined.

It is to be noted that a PTK with customized ablation surfaces is a promising instrument for treating focal anterior corneal pathologies and a recurrent erosion syndrome, independently of whether or not it is a focal pathology. It can be made easily available for laser treatment apparatuses.

The invention also includes embodiments, by which additional advantages arise.

In a further embodiment, the method can provide that the mask plane represents a plane, which is perpendicular to an irradiation direction of the laser onto the cornea in a rest position of a beam deflection device of the laser. This means that the virtual mask plane is perpendicular to an optical axis of the laser or of the treatment apparatus. Herein, the optical axis is the optical axis of the treatment apparatus in a rest position of the beam deflection device. The mask plane can be imagined as a virtual mask plane for example as situated immediately in front of the cornea. Herein, virtual means that the mask plane does not correspond to a real surface of the eye, but is a geometric construction aid of an image data processing for generating the control data, to determine the area to be irradiated using the virtual irradiation mask. Optionally, the virtual irradiation mask can be imagined as a shutter, shield or template in front of the eye, but which is only implemented or realized in a software for determining the area to be irradiated, without being configured as a shutter, shield or template in reality.

Further, points of the area to be irradiated are within the mask shape in a perpendicular projection onto the mask plane. Generally, the virtual irradiation mask is within an area, which results by an optical zone of the eye (or the pupil) in the perpendicular projection onto the mask plane, wherein a transition zone of the eye around the optical zone is to be incorporated.

By this definition of the mask plane, a particularly simple geometric setting of the virtual irradiation mask arises and it can be simply set in a software or an operating terminal.

In a further embodiment, the method can provide that the virtual irradiation mask is bounded by at least one closed boundary line, and the at least one closed boundary line comprises at least one vertex or at least two differently curved sections. This means that the virtual irradiation mask can have a shape deviating from a round shape and the closed boundary line can comprise at least one vertex, at least two differently curved sections or different radii of curvature or an elliptical shape with at least two different semi-axes. This has the advantage that the virtual irradiation mask can be particularly advantageously adapted to the area to be irradiated.

In a further embodiment, the method can provide that the virtual irradiation mask is bounded by at least two boundary lines, which are each closed and spaced from each other. This means that the boundary of the virtual irradiation mask can comprise multiple individual closed boundary lines, such that the virtual irradiation mask can, for example, be formed of multiple contiguous areas, which each comprise a closed boundary line, such as can, for example, comprise two separated circles situated next to each other. Further, the virtual irradiation mask can, for example, be bounded by two circles situated one within the other and thus represent a circular ring. Herein, spaced means that the boundary lines do not contact or intersect. This has the advantage that the virtual irradiation mask can be particularly advantageously adapted to the area to be irradiated.

In a further embodiment, the method can provide that the virtual irradiation mask can be selected from a plurality of preset geometric shapes. In other words, the shape of the virtual irradiation mask can be selected using a user interface, an operating terminal, a software or a computer program product, and can, for example, comprise various circular shapes, a ring shape, a crescent-like shape, a polygonal shape such as, for example, a triangular shape or a rectangular shape, a circular sector shape, a circular segment shape or an irregular shape, or can be composed of various lines and arcs. Further, the boundaries of the virtual irradiation mask can, for example, be effected by selecting the regions of the cornea by the user. In addition, preadjusted shapes can optionally be set by the system for the virtual irradiation mask, and an operating terminal can be formed such that the user only has to set the size and position of the preadjusted elements. Thus, this embodiment has the advantage that the virtual irradiation mask can be particularly well and simply adapted to the area to be irradiated.

In a further embodiment, the method can provide that the virtual irradiation mask is set such that it encompasses predetermined regions of the cornea or does not encompass predetermined regions of the cornea in the perpendicular projection of the cornea to the mask plane. In other words, the virtual irradiation mask can be set such that it includes certain regions such as, for example, a lesion in the perpendicular projection of the cornea to the mask plane such that areas of the cornea to be ablated are set. This means, the virtual irradiation mask can be set “ab-inclusio”.

Optionally, the virtual irradiation mask can be set such that it excludes predetermined regions of the cornea, this means that he mask is defined “ex-negativo”. For example, the mask can be set such that it excludes healthy areas of the cornea. Thus, these areas can be protected from an ablation. This has the advantage that the virtual irradiation mask can be particularly well adapted to the circumstances of the eye to be treated.

In a further embodiment, the method can provide that the virtual irradiation mask is set depending on preset patient data, in particular diagnostic data relating to the eye. In other words, the irradiation mask can be determined or set depending on patient data such that a, for example, pathologically altered area of the eye can be specifically irradiated by the laser of the treatment apparatus. The selection of the area to be irradiated and thereby the setting of the irradiation mask can be set depending on diagnostic data, which has been previously determined with respect to the eye. Such data can, for example, be Placido data, Scheimpflug data or optical coherence tomography data (OCT data). Further, the depth of a lesion can, for example, be determined by a corneal OCT. Further, an adapted ablation area can be set for a transepithelial PTK, which can, for example, be adapted to the accurate dimensions of a pathological alteration of the eye, such that the PTK can be adapted such that it only ablates the surface (the volume) of the pathology. Additionally, depths can be set in addition to the setting of the closed boundary line, which indicate a depth profile to be ablated. This has the advantage that the virtual irradiation mask can be particularly well adapted to the circumstances of the eye to be treated.

In a further embodiment, the method can provide that the virtual irradiation mask is set depending on a preset map of the eye in that map positions are associated with the virtual irradiation mask, in which an associated map value is greater than a value of a reference function in this map position. Herein, the map can be a perpendicular projection, for example, of the cornea to the mask plane, wherein the map value, for example, describes a local curvature of the cornea at this place. Thus, the virtual irradiation mask can be set based on the map positions in which a map value is greater than a value of a reference function at those positions. Such map positions are associated with the virtual irradiation mask. Herein, the reference function can, for example, be a constant or a polynomial function, can describe a curvature of the cornea, can be based on an ideal model or an average model of the eye, or can, for example, describe a thickness of the cornea based on pachymetric data.

It is to be noted that instead of the condition that the map value is greater than a value of the reference function at this place, the map value can also be less than or equal to a preset value of the reference function, in order to be associated with the virtual irradiation mask. For example, areas can be associated with the map, in which the curvature of the cornea is greater than 50 diopters. For example, areas can be associated with the virtual irradiation mask, in which the thickness of the epithelium is lower than the thickness of a normal epithelial profile. For example, areas can be associated with the map, in which the thickness of the cornea is thinner than 400 μm, or areas can be associated with the virtual irradiation mask, in which the thickness of the cornea is more than 400 μm. In addition, conditions can be combined using Boolean operators and/or brackets such that only values are, for example, associated with the virtual irradiation mask, in which the following condition is satisfied: [(the curvature is greater than 50 diopters) OR (the thickness of the epithelium at this place is less than the normal profile (Epi (x, y)))] AND [the pachymetric map (the thickness of the cornea at this place) is thicker than 400 μm].

In some circumstances, the virtual irradiation mask resulting from the map values has to be smoothed to define a larger or a contiguous region from individual points.

The advantage of this embodiment is in that the virtual irradiation mask can be extremely flexibly adapted to the given conditions of the eye.

In a further embodiment, the method can provide that the virtual irradiation mask is set depending on a local radius of curvature of the cornea and/or of the epithelial layer, depending on a local radius of curvature gradient of the cornea and/or of the epithelial layer, or depending on a local thickness of the cornea and/or of the epithelial layer. In other words, the virtual irradiation mask can be set such that it only includes regions, which are more severely or more weakly curved than a preset radius of curvature, or the radius of curvature gradient of which falls below or exceeds a preset radius of curvature gradient, or in which a thickness of the cornea exceeds or falls below a preset threshold value. Further, multiple of the above criteria can also be combined or linked with each other. Hereby, the advantage arises that the virtual irradiation mask can be particularly flexibly adapted to the circumstances of the eye.

In a further embodiment, the method can provide that the virtual irradiation mask is set depending on an image feature in an image captured by a camera. In other words, the virtual irradiation mask can be set depending on, for example, a brightness difference in an image or also a marking, for example, by gentian violet ink. Hereto, a scaling factor of the image of the cornea can be used to set the virtual irradiation mask. This embodiment is particularly advantageous if lesions cannot be clearly captured in a topography measurement or tomography measurement, such that the lesion surface can be marked with gentian violet ink in this case and thus an image of the cornea of the patient (which shows the marked areas) can be used for setting the virtual irradiation mask.

It is to be noted that the different above mentioned selection methods of the various embodiments can be combined with each other. This means that regions can be automatically selected and treatment depths are manually defined or that certain regions/volumes are automatically set and the user can manually add or remove further ones. The (semi) automatically selected regions (volumes) can be subsequently validated or for example improved by the user of the treatment apparatus before a treatment.

In a further embodiment, the method can provide that the image includes a result of a measurement of a topography of the cornea and/or the result of an optical coherence tomography measurement. The topography measurement of the cornea can, for example, be effected by a Placido measurement or a Scheimpflug measurement.

In a further embodiment, the method can provide that the irradiation treatment of the eye includes a treatment for removing dystrophy, in particular epithelial dystrophy, dystrophy of the Bowman's membrane and/or dystrophy of the stroma. Further, the irradiation treatment can also be effected for treating an apical scarring, a pellucid edge degeneration, a keratoconus and similar pathological alterations. Further examples for treatable dystrophies are a Reis-Bücklers corneal dystrophy, an epithelial basal membrane dystrophy, a Thiel-Behnke corneal dystrophy, a lattice corneal dystrophy, an Avelino dystrophy, a granular corneal dystrophy, a macular corneal dystrophy and a Schnyder corneal dystrophy. An advantage of this embodiment is in that the application of an adapted laser ablation surface in the treatment of focal stromal pathologies avoids an unnecessary epithelial and stromal ablation. This contributes to the fact that a visual restoration is faster effected and a procedure relating to a refractive power turns out less severe. This in particular relates to lesions at the edge of the cornea. In order to set an adapted ablation profile and thus an adapted virtual irradiation mask, a scaling factor of the image of the cornea can be known and be used.

According to a further embodiment, the method provides that the virtual irradiation mask is set using an operating terminal or the set virtual irradiation mask has to be confirmed by a user of the treatment apparatus. In other words, the treatment apparatus can include an operating terminal and the virtual irradiation mask can be set using the operating terminal. If a virtual irradiation mask is set based on image features or the like by the method, a confirmation of the virtual irradiation mask can also be effected by a user of the treatment apparatus before an irradiation treatment. A processing software, which can execute the method, can ascertain the exact shape, size and the location of a lesion based on an image of the eye in that the pupil or the corneal vertex is for example contemplated as reference points. Herein, a scaling factor can be applied according to need.

Some lesions can represent a depression, while others represent an elevation and some can even represent both. All of the determined dimensions, including distances and the angles thereof and also the depth of a lesion, can allow setting a size and a location of a surface to be ablated. The associated depth for each partial region of the area to be irradiated can be automatically or semi-automatically associated from a corneal OCT measurement in that regions with a more severe scattering or a hyper-reflection are determined. The areas to be irradiated, which are derived from a corneal OCT measurement, can be smoothed to define a larger or a contiguous region from individual points.

In a further embodiment, the method can provide that a position course of the treated eye is ascertained during the irradiation or during the treatment of the area to be irradiated. This means that a so-called eye tracker can be used, which can determine and quantify a movement, a position or pose change of the eye. Further, the irradiation position of the laser can be corrected depending on the determined movement, position or pose change of the eye during the irradiation to compensate for or offset such a deviation. This means that a deviation of the eye by a tremor or the like can be compensated for and the irradiation can be more safely effected. This allows using an adapted ablation surface for a PTK treatment, which has an improved precision due to tracking the eye movement, the position or pose change.

In contrast to a manually decentered PTK with a small diameter or to a use of a polyvinyl alcohol disk with a punched hole as known in the prior art, an improved precision can be achieved in this embodiment due to the use of the eye tracker.

This object is solved by the method according to the invention, the control device according to the invention, the treatment apparatus according to the invention, the computer program product according to the invention as well as the computer-readable medium according to the invention. Advantageous embodiments comprising suitable developments of the invention are specified in the respective dependent claims, wherein advantageous embodiments of the method are to be regarded as advantageous embodiments of the control device, the treatment apparatus, the computer program product and the computer-readable medium and vice versa.

The control data can include a respective dataset for positioning and/or for focusing individual laser pulses in or on the cornea. Additionally or alternatively, a respective dataset for adjusting at least one beam deflection device for beam guidance and/or beam shaping and/or beam deflection and/or beam focusing of a laser beam of the respective laser can be included in the control data.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as, for example, a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed using the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially remove, separate, atrophy or ablate a predefined corneal volume with predefined interfaces of a human or animal eye by cauterization or by optical breakdown, in particular at least partially to separate it by photodisruption and/or to ablate corneal layers by (photo) ablation and/or to cause a laser-induced refractive index change in the cornea and/or the eye lens.

A further aspect of the invention relates to a computer program product. The computer program product includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program product and the commands thereof, respectively, are stored. For executing the computer program product, a computer or a computer cluster can access the computer-readable medium and read out the content thereof. The storage medium is, for example, formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the developments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous embodiments based on the figures. The features or feature combinations of the embodiments described in the following can be present in any combination with each other and/or the features of the embodiments. This means, the features of the embodiments can supplement and/or replace the features of the embodiments and vice versa. Thus, embodiments are also to be regarded as encompassed and disclosed by the invention, which are not explicitly shown or explained in the figures, but arise from and can be generated by separated feature combinations from the embodiments and/or embodiments. Thus, embodiments are also to be regarded as disclosed, which do not comprise all of the features of an originally formulated claim or extend beyond or deviate from the feature combinations set forth in the relations of the claims. To the embodiments, there shows:

FIG. 1 depicts a schematic representation of a treatment apparatus according to an exemplary embodiment;

FIG. 2 depicts a schematic representation of the relation between mask plane and a cornea of an eye;

FIG. 3a depicts a schematic representation of a mask plane and a boundary line; and

FIG. 3b depicts a schematic representation of a mask plane and a boundary line.

### DETAILED DESCRIPTION

In the figures, identical or functionally identical elements are provided with the same reference characters.

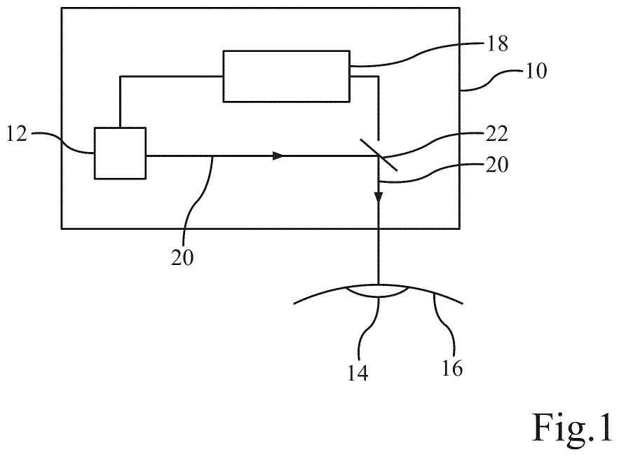

FIG. 1 shows a schematic representation of a treatment apparatus 10 with an ophthalmological laser 12 for treating a cornea of an eye 16, for example, by photodisruption and/or ablation. For treating the cornea, treatment positions within the area 14 to be treated are preset in the control data, in which a tissue to be cauterized or to be ablated is, for example, present. Besides the laser 12, a control device 18 for the laser 12 can be formed such that it can emit pulsed laser pulses, for example, in a predefined pattern. Alternatively, the control device 18 can be a control device 18 external with respect to the treatment apparatus 10.

Furthermore, FIG. 1 shows that the laser beam 20 generated by the laser 12 is deflected towards the eye 16 by a beam deflection device 22 such as, for example, a rotation scanner. The beam deflection device 22 is also controlled by the control device 18 to treat the eye 16.

The illustrated laser 12 can preferably be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. In addition, the control device 18 comprises a storage device for storing at least one control dataset, wherein the control dataset or datasets include(s) control data for positioning and/or for focusing individual laser pulses in the area 14 to be treated. Herein, the storage device can be a part of the control device 18 or the storage device can be provided as an external memory, in particular in the form of a computer cluster (cloud). The position data and/or focusing data of the individual laser pulses, in particular for a laser treatment, can be generated based on predetermined measurements, for example from a previously measured topography and/or pachymetry and/or the morphology of the cornea or the optical visual disorder correction to be generated.

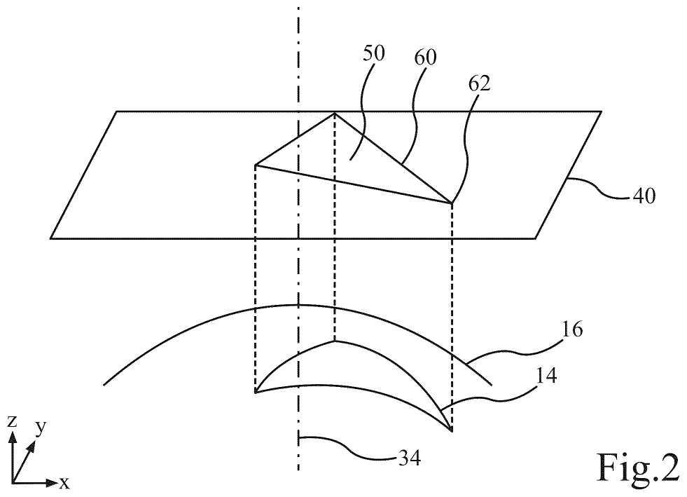

FIG. 2 shows a schematic representation of a relation between a mask plane and the eye. In particular, FIG. 2 shows the eye 16, an optical axis 34 of the laser or of the treatment apparatus 10, an area 14 to be irradiated, a perpendicular projection onto the mask plane 40 and the virtual irradiation mask 50 with the boundary line 60. Herein, the boundary line 60 comprises a vertex 62. As is apparent from FIG. 2, the mask plane represents a plane, which is perpendicular to the optical axis 34 of the laser or the treatment apparatus in a rest position of a beam deflection device 22 of the laser 12. Herein, the optical axis 34 of the laser is also the optical axis 34 of the treatment apparatus 10 in the rest position of the beam deflection device 22. The area 14 to be irradiated can have a complex shape on or in the cornea, and is within the virtual irradiation mask 50 in the perpendicular projection onto the mask plane 40. Subsequently, the laser 12 can emit laser pulses to the area 14 to be irradiated such that the area 14 to be irradiated is irradiated with laser pulses. In order to direct the laser 12 to the points to be irradiated, the beam deflection device 22 is suitably controlled. In particular, the area 14 to be irradiated can be a curved surface or a 3D volume, the area 14 to be irradiated can be a pathologically altered area, which is set automatically or by the user depending on examination data of the cornea.

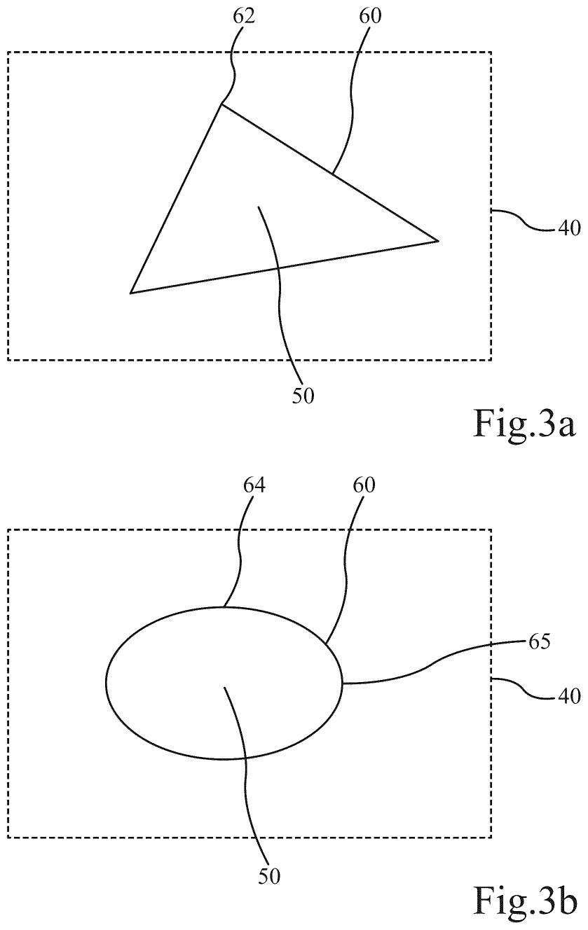

FIG. 3a additionally shows a schematic representation of a mask plane 40 with a virtual irradiation mask 50, which comprises a boundary line 60. Herein, the boundary line 60 comprises at least one vertex. FIG. 3b also illustrates a schematic representation of a mask plane and a boundary line 60, which bounds a virtual irradiation mask 50. In the example according to FIG. 3b, the boundary line 60 comprises a first curved section 64 and a second curved section 65. Further, the boundary line 60 is obviously closed in this case too. In the example of FIG. 3b, the boundary line 60 comprises at least two differently curved sections 64, 65. This means, the boundary line 60 has a shape, which is not round, but is, for example, elliptic with at least two different radii of curvature or semi-axes as in this example.

Generally, the virtual irradiation mask 50 can be selected from a plurality of preset geometric shapes. For example, this can be effected using a user interface, and the user can, for example, have a crescent shape, a polygonal shape such as, for example, a triangle (as shown in FIG. 3a), a ring shape, a sector shape, a segment shape, an irregular shape, multiple single shapes and so on. Further, the virtual irradiation mask 50 can be set such that it encompasses or includes predetermined regions of the cornea in the perpendicular projection of the cornea to the mask plane, or does not encompass or excludes predetermined regions of the cornea. Further, the virtual irradiation mask 50 can be set depending on preset patient data, that is diagnostic data relating to the eye. Herein, a virtual irradiation mask 50 can, for example, be set directly from the patient data or it is set depending on a preset map of the eye. Map positions in the map are associated with the virtual irradiation mask 50, in which an associated map value is greater or less than or equal to a value of a reference function in this map position. Herein, the reference function can be a constant or can be a function depending on the map position. For example, curvature values, which are greater than for example 50 diopters, can be associated with the virtual irradiation mask 50. Further, values of the epithelial map can be associated with the virtual irradiation mask 50, in which values of the epithelial map are less than the normal profile of an average epithelium, can be associated with the virtual irradiation mask 50. Further, values of a pachymetric map can be used, and places, at which the values are less than 400 μm, can be associated with the virtual irradiation mask 50. It is obvious that these conditions can be combined with each other using Boolean operators and/or brackets such that only values are for example associated with the virtual irradiation mask 50, at which the curvature is greater than 50 diopters or the thickness of the epithelium at this place is less than the normal profile (Epi (x, y)) than the normal profile, and the pachymetric map (the thickness of the cornea at this place) is thicker than 400 μm. In some circumstances, this mask has to be smoothed to define a larger or a contiguous region from individual points. Further, the virtual irradiation mask 50 can also be set depending on an image feature in an image captured by a camera. Herein, brightness differences in an image can for example be used to derive a virtual irradiation mask 50, or colored markings such as for example a marking with gentian violet ink can also be used, to derive and set a virtual irradiation mask 50. In addition, the image can be a result of a measurement of a topography of the cornea, for example a Placido measurement or a Scheimpflug measurement, and/or the result of an optical coherence tomography measurement.

In addition, the virtual irradiation mask 50 can be set such that it is suitable for treating a removal of a dystrophy, in particular an epithelial dystrophy, a dystrophy of the Bowman's membrane and/or a dystrophy of the stroma. The virtual irradiation mask 50 can be set using an operating terminal or the virtual irradiation mask 50, which is automatically set by the method, can be confirmed by a user of the treatment apparatus.

The setting of a virtual irradiation mask 50 additionally allows that a position course of the treated eye is set during the irradiation or during the treatment of the area to be irradiated, for example, using an eye tracker, and the irradiation position of the laser is corrected during the irradiation to compensate for a position deviation of the eye 16.

Overall, the embodiments show, how a method for providing control data for an ophthalmological laser of a treatment apparatus, a control device therefor, a treatment apparatus, a computer program product and a computer-readable medium can be provided.

### LIST OF REFERENCE CHARACTERS:

10 Treatment apparatus
12 laser
14 area to be irradiated
16 eye
18 control device
20 laser beam
22 beam deflection device
34 optical axis
40 mask plane
50 virtual irradiation mask
60 boundary line
62 vertex
64 curved section
65 curved section

1. A method for providing control data for an ophthalmological laser of a treatment apparatus for treating a human or animal eye, wherein the method comprises the following steps performed by a control device:
setting an area to be irradiated on or in a cornea of the eye to be treated for performing an irradiation treatment with the laser,
setting a virtual irradiation mask in a mask plane,
wherein the area to be irradiated is situated within the virtual irradiation mask in a perpendicular projection onto the mask plane, and
providing control data for the laser by the control device, wherein the control data includes coordinates of the area to be irradiated, such that the laser emits laser pulses to the area to be irradiated during the treatment.

2. The method according to claim 1, wherein the mask plane represents a plane, which is perpendicular to an irradiation direction of the laser to the cornea in a rest position of a beam deflection device of the laser.

3. The method according to claim 1, wherein the virtual irradiation mask is bounded by at least one closed boundary line, and the at least one closed boundary line comprises at least one vertex or at least two differently curved sections.

4. The method according to claim 1, wherein the virtual irradiation mask is bounded by at least two boundary lines, which are each closed and spaced apart from each other.

5. The method according to claim 1, wherein the virtual irradiation mask is selected from a plurality of preset geometric shapes.

6. The method according to claim 1, wherein the virtual irradiation mask is set such that it includes predetermined regions of the cornea in a perpendicular projection of the cornea onto the mask plane.

7. The method according to claim 1, wherein the virtual irradiation mask is set depending on preset patient data.

8. The method according to claim 1, wherein the virtual irradiation mask is set depending on a preset map of the eye, wherein map positions, having an associated map value greater than a value of a reference function at those map positions, are associated with the virtual irradiation mask.

9. The method according to claim 1, wherein the virtual irradiation mask is set depending on a local radius of curvature of the cornea and/or of an epithelial layer, depending on a local radius of curvature gradient of the cornea and/or of the epithelial layer, or depending on a local thickness of the cornea and/or of the epithelial layer.

10. The method according to claim 1, wherein the virtual irradiation mask is set depending on an image feature in an image captured by a camera.

11. The method according to claim 10, wherein the image includes a result of a measurement of a topography of the cornea and/or the result of an optical coherence tomography measurement.

12. The method according to claim 1, wherein the irradiation treatment of the eye includes a treatment for removing dystrophy.

13. The method according to claim 1, wherein the virtual irradiation mask is set by an operating terminal or the set virtual irradiation mask has to be confirmed by a user of the treatment apparatus.

14. The method according to claim 1, wherein a position course of the treated eye is ascertained during the irradiation of the area to be irradiated, and the irradiation position of the laser is corrected during the irradiation to compensate for a position deviation of the eye.

15. The method according to claim 1, further including the following steps:
transferring the provided control data to the laser of the treatment apparatus, and
controlling the laser with the control data.

16. A control device, which is configured to perform the method according to claim 1.

17. A treatment apparatus with at least one ophthalmological laser for treating a cornea of a human or animal eye by cauterization or by optical breakdown, in particular by photodisruption and/or photoablation, and the control device according to claim 16.

18. (canceled).

19. A computer-readable medium for storing a computer program product thereon, the computer program product comprising commands which cause a treatment apparatus to execute the method according to claim 1.

20. The method according to claim 1, wherein the virtual irradiation mask is set such that it does not include predetermined regions of the cornea in a perpendicular projection of the cornea onto the mask plane.

21. The method according to claim 12, wherein the treatment for removing dystrophy includes treatment of epithelial dystrophy, dystrophy of a Bowman's membrane, and/or dystrophy of a stroma.
